(19) United States
(12) Patent Application Publication
(10) Pub. No.: US 2019/0357596 A1
(43) Pub. Date: Nov. 28, 2019

(54) VAPOR PROVISION SYSTEM

(71) Applicant: BRITISH AMERICAN TOBACCO (INVESTMENTS) LIMITED, London (GB)

(72) Inventors: Kevin David BLICK, London (GB); Alfred Vincent SPENCER, LONDON (GB); Dominic WOODCOCK, LONDON (GB)

(21) Appl. No.: 16/484,966

(22) PCT Filed: Jan. 30, 2018
(86) PCT No.: PCT/GB2018/050266

§ 371 Date: Aug. 9, 2019

(30) Foreign Application Priority Data
Feb. 10, 2017 (GB) 1702207.0

(51) Int. Cl. A24F 47/00; A61M 11/00; A61M 11/04

(52) U.S. Cl. CPC A24F 47/008; A61M 11/001; F17C 9/02; A61M 2205/8206; A61M 11/042

(57) ABSTRACT

A vapor provision system configured to generate vapor with a selectable degree of visibility for inhalation and subsequent exhalation by a user, including a first vapor precursor material for generating a vapor having a first degree of visibility; a second vapor precursor material for generating a vapor having a second degree of visibility, wherein the first degree of visibility is greater than the second degree of visibility such that vapor generated from the second vapor precursor material is less visible than vapor generated from the first vapor precursor material; at least one vaporizer operable to generate vapor from the first vapor precursor material and the second vapor precursor material; and control circuitry configured to control the at least one vaporizer to generate vapor from the first vapor precursor material and the second vapor precursor material in a selectable ratio to provide a vapor having a correspondingly selectable degree of visibility.

### PRIORITY CLAIM

The present application is a National Phase entry of PCT Application No. PCT/GB2018/050266, filed Jan. 30, 2018, which claims priority from GB Patent Application No. 1702207.0, filed Feb. 10, 2017, each of which is hereby fully incorporated herein by reference.

### TECHNICAL FIELD

The present disclosure relates to vapor provision systems such as nicotine delivery systems (e.g. electronic cigarettes and the like), and in particular to providing users with the ability to modify the visibility of vapor generated by vapor provision systems.

### BACKGROUND

Electronic vapor provision systems such as electronic cigarettes (e-cigarettes) generally contain a vapor precursor material, such as a reservoir of a source liquid containing a formulation, typically comprising a base liquid with additives such as nicotine and often flavorants, and/or a solid material such as a tobacco-based product, from which a vapor is generated for inhalation by a user, for example through heat vaporization. Thus, a vapor provision system will typically comprise a vapor generation chamber containing a vaporizer, e.g. a heating element, arranged to vaporize a portion of precursor material to generate a vapor in the vapor generation chamber. As a user inhales on the device and electrical power is supplied to the vaporizer, air is drawn into the device through inlet holes and into the vapor generation chamber where the air mixes with the vaporized precursor material to form an aerosol. There is a flow path connecting the vapor generation chamber with an opening in the mouthpiece so the incoming air drawn through the vapor generation chamber continues along the flow path to the mouthpiece opening, carrying some of the vapor with it, and out through the mouthpiece opening for inhalation by the user.

Vapor provision systems may comprise a modular assembly including both reusable and replaceable cartridge parts. Typically a cartridge part will comprise the consumable vapor precursor material and/or the vaporizer, while a reusable device part will comprise longer-life items, such as a rechargeable battery, device control circuitry, activation sensors and user interface features. The reusable part may also be referred to as a control unit or battery section and replaceable cartridge parts that include both a vaporizer and precursor material may also be referred to as cartomizers.

Cartridges are electrically and mechanically coupled to a control unit for use, for example using a screw thread or bayonet fixing with appropriately engaging electrical contacts. When the vapor precursor material in a cartridge is exhausted, or the user wishes to switch to a different cartridge having a different vapor precursor material, a cartridge may be removed from the control unit and a replacement cartridge attached in its place.

Thus a vapor provision system will generate a vapor for inhalation by a user to deliver a desired taste/smell sensation and/or deliver nicotine. The vapor exhaled by a user following inhalation is often visible. For some users this is a desired characteristic of the vapor, for example a user may find that exhaling a cloud of visible vapor is an enjoyable part of their experience, whereas for other users, or in some use scenarios, this may not be a desired characteristic of the vapor, for example a user may not wish not to draw attention to their use of the vapor provision system. Some users may want to generate a more visible vapor on some occasions, for example when at home, and a less visible vapor on other occasions, for example when in a public area. In view of this, the inventors have recognized a desire to provide approaches for modifying the visibility of vapor generated by vapor provision systems.

### SUMMARY

According to a first aspect of certain embodiments there is provided a vapor provision system configured to generate vapor with a selectable degree of visibility for inhalation and subsequent exhalation by a user; wherein the system comprises: a first vapor precursor material for generating a vapor having a first degree of visibility; a second vapor precursor material for generating a vapor having a second degree of visibility, wherein the first degree of visibility is greater than the second degree of visibility such that vapor generated from the second vapor precursor material is less visible than vapor generated from the first vapor precursor material; at least one vaporizer operable to generate vapor from the first vapor precursor material and the second vapor precursor material; and control circuitry configured to control the at least one vaporizer to generate vapor from the first vapor precursor material and the second vapor precursor material in a selected ratio to provide a vapor having a correspondingly selected degree of visibility.

According to another aspect of certain embodiments there is provided vapor provision means for generating a vapor with a selectable degree of visibility for inhalation and subsequent exhalation by a user; comprising: a first vapor precursor material adapted for generating a vapor having a first degree of visibility; a second vapor precursor material for generating a vapor having a second degree of visibility, wherein the first degree of visibility is greater than the second degree of visibility such that vapor generated from the second vapor precursor material is less visible than vapor generated from the first vapor precursor material; vaporizing means for generating vapor from the first vapor precursor material and the second vapor precursor material; and control means for controlling the vaporizing means to generate vapor from the first vapor precursor material and the second vapor precursor in a selected ratio to provide a vapor having a correspondingly selected degree of visibility.

According to another aspect of certain embodiments there is provided a method of generating a vapor with a selectable degree of visibility for inhalation and subsequent exhalation by a user; the method comprising: providing a vapor provision system comprising: a first vapor precursor material for generating a vapor having a first degree of visibility; a second vapor precursor material for generating a vapor having a second degree of visibility, wherein the first degree of visibility is greater than the second degree of visibility such that vapor generated from the second vapor precursor material is less visible than vapor generated from the first vapor precursor material; and at least one vaporizer operable to generate vapor from the first vapor precursor material and the second vapor precursor material; and controlling the at least one vaporizer to generate vapor from the first vapor precursor material and the second vapor precursor in a selected ratio to provide a vapor having a correspondingly selected degree of visibility.

These and further aspects of certain embodiments are set out in the appended independent and dependent claims. It will be appreciated that features of the dependent claims may be combined with each other and features of the independent claims in combinations other than those explicitly set out in the claims. Furthermore, the approaches described herein are not restricted to specific embodiments such as the examples set out below, but include and contemplate any appropriate combinations of features presented herein. For example, a vapor provision system may be provided in accordance with approaches described herein which includes any one or more of the various features described below as appropriate.

### BRIEF DESCRIPTION OF THE DRAWINGS

Embodiments will now be described, by way of example only, with reference to the accompanying drawings, in which:

FIG. 1 represents in highly schematic cross-section an aerosol provision system in accordance with certain embodiments of the disclosure.

FIG. 2 represents a partially exploded perspective view of the aerosol provision system of FIG. 1.

FIG. 3 is a flow diagram schematically representing a method of operating the aerosol provision system of FIGS. 1 and 2 in accordance with certain embodiments of the disclosure.

FIG. 4 represents in highly schematic cross-section an aerosol provision system in accordance with certain other embodiments of the disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to vapor provision systems, which may also be referred to as aerosol provision systems, such as e-cigarettes. Throughout the following description the term “e-cigarette” or “electronic cigarette” may sometimes be used; however, it will be appreciated this term may be used interchangeably with vapor provision system and electronic vapor provision system. Furthermore, and as is common in the technical field, the terms “vapor” and “aerosol”, and related terms such as “vaporize”, “volatilize” and “aerosolize”, may also be used interchangeably.

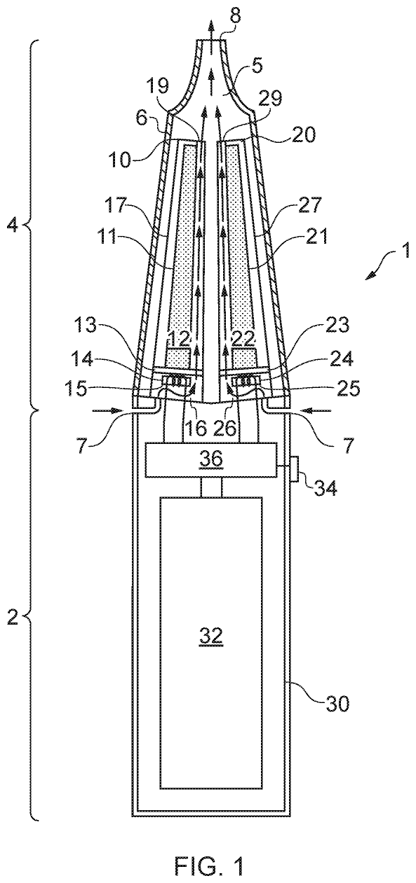

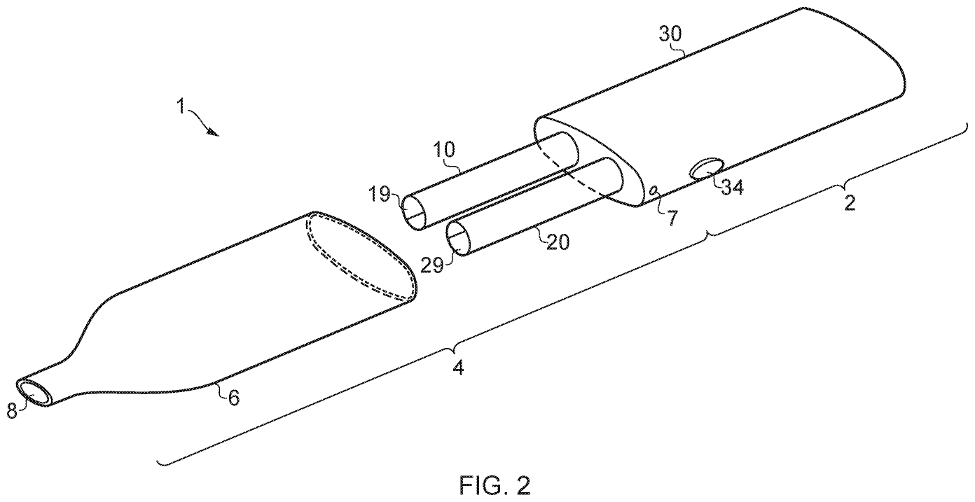

FIGS. 1 and 2 are respectively schematic cross-sectional and perspective views of an example e-cigarette 1 in accordance with some embodiments of the disclosure. The view of FIG. 2 is partially exploded in showing the e-cigarette 1 with a mouthpiece cover 6 separated from the remainder of the system/device. The e-cigarette 1 may be considered to comprise two main components, namely a control unit 2 and a vapor generation assembly 4. One significant aspect of the electronic cigarette 1 represented in FIG. 1 is that the vapor generation assembly 4 comprises multiple cartridges, in this case two cartridges (other example implementations may comprise more than two cartridges/sources of vapor precursor material). The control unit 2 is configured to selectively activate vapor generation from one or other or both cartridges, for example in response to user settings, to allow a user to choose whether to generate vapor from one or other or both cartridges (and in what proportion).

Significantly, and as discussed further below, in accordance with certain embodiments of the disclosure, different ones of the cartridges of the electronic cigarette 1 are arranged to generate vapor having different degrees of visibility, thereby allowing a user to readily select a degree of visibility associated with vapor generated by the electronic cigarette 1 by selecting which cartridge to use to generate vapor, for example on a puff-by-puff basis. In addition to generating vapor from one or other cartridge, the electronic cigarette 1 in this example is configured to also allow vapor to be generated from both cartridges at the same time to generate a vapor mixture having a degree of visibility that is between that associated with the individual cartridges. More generally, different degrees of vapor visibility/opacity/transparency may be obtained by generating vapor from the different cartridges in different relative amounts/ratios/proportions.

Thus, the vapor assembly 4 comprises a first cartridge 10 for generating a vapor having a first degree of visibility and a second cartridge 20 for generating a vapor having a second degree of visibility. It will be assumed for this example the first degree of visibility is greater than the second degree of visibility, that is to say the first cartridge is associated with generating a vapor which is more visible (higher opacity/less transparency) than the second cartridge, but this could equally be the other way around. For ease of explanation and simplicity in terminology in the following description, the vapor associated with the first cartridge may sometimes be referred to herein as visible vapor while the vapor associated with the second cartridge may sometimes be referred to herein as less-visible vapor. It will be appreciated that the specific opacity associated with a vapor will depend on a range of factors, and not just the composition of the vapor precursor material in the cartridge. For example, the opacity associated with a vapor from a given electronic cigarette may also be expected to depend on the amount of power used to generate the vapor, the ambient air temperature and moisture content, and the manner in which the vapor is dispersed, for example by fast or slow exhalation. Accordingly, references herein to visible and less-visible vapors are to be interpreted as referring to the relative degree of visibility/opacity associated with a vapor generated from different cartridges under comparable conditions (e.g. similar power supply, air temperature, method of inhalation/exhalation). In this regard, the most significant factor contributing to the degree of visibility of a vapor will be in the nature/composition of the source material.

Referring again to FIGS. 1 and 2, the first and second cartridges 10, 20 are removably mounted to the control unit 2 in an appropriate manner (e.g. using a conventional bayonet fixing, screw thread or friction-fit fixing). In this example the cartridges are mounted in a generally side-by-side configuration. The mouthpiece cover 6, which is generally hollow, is also removably coupled to the control unit 2, and again this may be achieved in accordance with any conventional coupling/mounting technique, e.g. a snap-fit fixing. In the cross-sectional representation of FIG. 1 the mouthpiece cover 6 is shown coupled to the control unit 2 for normal use in which it covers the first and second cartridges. In the perspective view of FIG. 2, the mouthpiece cover 6 is shown separated from the control unit 2, for example to provide access to the cartridges 10, 20 to allow them to be replaced. The mouthpiece cover 6 is provided with a tapered end having an opening 8 that defines a vapor outlet through which a user may inhale vapor generated by the electronic cigarette 1 during use. An interior space within the mouthpiece cover 6 between the cartridges 10, 20 and the vapor outlet 8 defines a region 5 through which vapor generated from the respective cartridges 10, 20 passes, and may be mixed when both cartridges are used to generate vapor simultaneously, for inhalation by a user through the mouthpiece outlet 8.

The first and second cartridges in this example are substantially identical in terms of their structure and operation, but differ in terms of the visibility characteristics of the vapor they produce resulting from differences in the vapor precursor material used by the two cartridges in terms of the visibility (opacity) of vapor they produce.

A liquid vapor precursor material for an electronic cigarette will typically comprise a base liquid formulation, which makes up the majority of the liquid, with additives for providing desired flavor/smell/nicotine delivery characteristics to the base liquid. Different base liquids are associated with vapors having different degrees of visibility. For example, a typical base liquid may comprise a mixture of propylene glycol (PG) and vegetable glycerol (VG), and a base liquid having a relatively high proportion of VG will typically create a more visible (less transparent) vapor than an otherwise corresponding base liquid having a relatively low proportion of VG.

Thus, in accordance with certain embodiments of the disclosure, the vapor precursor material for the first cartridge comprises a first base liquid and the vapor precursor material for the second cartridge comprises a second base liquid, wherein the first base liquid and the second base liquid are associated with vapors having different degrees of visibility. The additives to the base liquids for the respective cartridges may be the same such that the difference between the vapor generated by the different cartridges is only from the use of different base liquids. Alternatively, the additives for the respective cartridges may be different such that in addition to generating vapors of different visibility/opacity, the two cartridges are also associated with vapors having different flavors and/or smells and/or amounts of nicotine.

For the sake of a concrete example, it is assumed here the first base liquid (used in the first cartridge) comprises 80% VG and 20% PG, which is associated with generating a relatively visible vapor, and the second base liquid (used in the second cartridge) comprises 80% PG and 20% VG, which is associated with generating a relatively invisible (less-visible) vapor. For completeness, and although this is not significant to the principles described herein, it is further assumed for this example that the vapor precursor materials for the first cartridge and the second cartridge comprise the same additives for providing the same flavor of vapor and the same level of nicotine delivery. That is to say, in accordance with this example, it is assumed that the only difference between the vapors generated by the first and second cartridges are from the use of the different base liquids in these cartridges giving rise to different degrees of vapor visibility/opacity. Despite the different cartridges in this particular example having the same additives (e.g. in terms of flavorant/nicotine strength), it will be appreciated a user may still perceive a different taste/smell/mouth feel for the vapor form each cartridge because of the sensorial effects of the different base liquids, but this is not significant to the principles described herein.

The difference in visibility for vapors associated with the different cartridges may be different in different implementations. For example in some examples there may be a greater difference in opacity between the vapors from the different cartridges than in other examples. More generally, the opacity of vapor from the cartridge associated with the more-visible vapor may be greater than the opacity of vapor from the cartridge associated with the less-visible vapor by at least a factor selected from the group comprising 1.5, 2, 3, 4, 5, 6, 7, 8, 9, 10, or more for different implementations. The relative degrees of opacity for vapors from different cartridges may, for example, be established for different base liquid formulations through empirical testing. In this regard, a measure of the characteristic opacity of vapors can be established in accordance with conventional techniques for measuring/defining optical transparency/visibility. For example, this may be based on measuring light transmission/scattering/absorption associated with the respective vapors in a test environment and characterizing the opacity in terms of the amount of transmission and/or scattering and/or absorption for a given path length through the vapor.

In view of the structural similarity between the two cartridges 10, 20 (i.e. in this example the difference is only in the specific formulation of vapor precursor material contained within the cartridges), the cartridges are described in more detail below with a focus on the first cartridge 10 associated with the more-visible vapor, but it will be understood that the same description applies for the second cartridge 20 associated with the less-visible vapor.

Thus, the first cartridge 10 comprises a cartridge housing 17, which in this example is formed of a plastics material. The housing 17 supports other components of the cartridge and also provides a mechanical interface with the control unit 2. The manner by which the cartridge 10 mounts to the control unit is not significant to the principles described herein, but for the sake of a concrete example is assumed here to comprise a screw thread fitting (not represented in FIG. 1).

The cartridge housing 17 is generally circularly symmetric about a longitudinal axis with a tapering profile so it reduces in cross section with increasing distance from the end of the cartridge 10 which couples to the control unit 2. In this example the cartridge has a length of around 4 cm and a diameter that tapers down approximately linearly from around 1 cm to 0.7 cm over this length. However, it will be appreciated the specific geometry, and more generally the overall shapes involved, may be different in different implementations.

Within the cartridge housing 17 is a liquid reservoir 11 that contains the vapor precursor material in the form of the first source liquid 12 discussed above. The liquid reservoir 11 in this example comprises the majority of the interior volume of the cartridge 10. The liquid reservoir 11 generally conforms to the interior of the housing 17 in having a tapering circular cross section, but having a flat face running longitudinally along one side to create a space between an outer wall of the reservoir 11 and an inner wall of the housing 17. This defines an air path through the cartridge through which vapor generated in the cartridge is drawn during use towards an opening 19 in the end of the cartridge into the chamber/region 5 within the mouthpiece cover 6. This air-path through the cartridge is schematically represented by the series of arrows indicating airflow through the vapor provision system 1 during use. The reservoir 11 may be formed in accordance with conventional techniques, for example comprising a molded plastics material.

An end of the reservoir 11 opposite to the cartridge outlet 19 is defined by a porous ceramic disc 13 such that source liquid 12 within the reservoir 11 may seep through the ceramic disc 13.

Adjacent the ceramic disc 13 on the outside of the reservoir 11 is a vaporizer (atomizer) comprising a wick 14 and heater 15. The wick and heater are arranged in a space within the cartridge housing 17 that defines a vaporization chamber 16 for the cartridge 10.

Source liquid which has seeped through the ceramic disc 13 may infiltrate the wick 14 through surface tension/capillary action. The heater 15 in this example comprises an electrically resistive wire coiled around the wick 14. Electrical power may be supplied to the heater 15 to vaporize an amount of source liquid (vapor precursor material) drawn to the vicinity of the heater 15 by the wick 14. In this example the heater 15 comprises a nickel chrome alloy (Cr20Ni80) wire and the wick 14 comprises a glass fiber bundle, but it will be appreciated that the specific vaporizer configuration is not significant to the principles described herein.

The rate at which source liquid is vaporized by the vaporizer will depend on the amount of power supplied to the heater 15. Accordingly, electrical power can be applied to the heater to selectively generate vapor from the source liquid 12 in the cartridge 10, and furthermore, at least in some example implementations, the rate of vapor generation can be controlled by adjusting the power supplied to the heater 15, for example through pulse width and/or frequency modulation techniques.

As already mentioned above, the second cartridge 20 has the same overall structure as the first cartridge 10 (indeed the two cartridges are interchangeable in terms of how they may be mounted within the vapor provision system in this example). Accordingly, and as for the first cartridge 10, the second cartridge 20 also comprises a cartridge housing 27, a liquid reservoir 21, in this case containing the second source liquid 22 associated with less-visible vapor, a cartridge outlet 29, a ceramic disc 23, a wick 24, a heater 25 and a vaporization chamber 26. These elements of the second cartridge are arranged in the manner described above for the corresponding elements of the first cartridge 10. In other example implementations the different cartridges may have different structures and/or sizes.

The control unit 2 comprises an outer housing 30, a battery 32 for providing operating power for the electronic cigarette, control circuitry 36 for controlling and monitoring the operation of the electronic cigarette and a user input button 34. The battery 32 is rechargeable and may be of a conventional type, for example of the kind normally used in electronic cigarettes and other applications requiring provision of relatively high currents over relatively short periods. Similarly, the user input button 34 may be a conventional input device, for example a mechanical button switch or capacitive (touch) sensor.

The outer housing 30 may be formed, for example, from a plastics or metallic material and in this example has a generally elliptical/oval cross sectional with a width (in the plane of FIG. 1) of around 1.5 to 2 times its thickness (perpendicular to the plane of FIG. 1). For example, the electronic cigarette may have a width of around 3 cm and a thickness of around 2 cm. The mouthpiece cover 6 discussed above has an outer form which generally conforms to the outer form of the control unit 2 where they meet to provide a relatively uniform and smooth appearance for the electronic cigarette 1 as a whole. The end of the mouthpiece cover 6 defining the vapor outlet 8 is tapered down to around one third or so of its dimensions at the end that couples to the control unit 2 (e.g. to around 1 cm wide and 0.6 cm thick). The control unit 2 and mouthpiece cover 6 in this example both have a length of around 5 cm such that the assembled electronic cigarette has a length of around 10 cm. However, and as already noted, it will be appreciated that the overall shape and scale of an electronic cigarette implementing an embodiment of the disclosure is not significant to the principles described herein.

The control circuitry 36 is suitably configured/programmed to provide functionality in accordance with embodiments of the disclosure as described herein, as well as for providing conventional operating functions of the electronic cigarette in line with the established techniques for controlling such devices. Thus the control circuitry may be considered to logically comprise a number of different functional blocks, for example a functional block for controlling the supply of power from the battery 32 to the heater 15 in the first cartridge 10, a functional block for controlling the supply of power from the battery 32 to the heater 25 in the second cartridge 20, a functional block for controlling operational aspects of the device in response to user input using the input button 34, for example configuration settings, as well as other functional blocks associated with the normal operation of electronic cigarettes and functionality in accordance with the principles described herein. It will be appreciated the functionality of these logical blocks may be provided in various different ways, for example using a single suitably programmed general purpose computer, or suitably configured application-specific integrated circuit(s)/circuitry. As will be appreciated the electronic cigarette will in general comprise various other elements associated with its operating functionality, for example a port for charging the battery, such as a USB port, and these may be conventional and are not shown in the figures or discussed in detail in the interests of brevity.

The control circuitry 36 is configured to control the supply of electrical power from the battery 32 to the heaters 15, 25 in the respective cartridges 10, 12 so as to selectively generate a vapor from one or other or both cartridges for inhalation by a user. Electrical power is supplied to the respective heaters via contacts established across the interface between the respective cartridges 10, 20 and the controller unit 2, for example through sprung/pogo pin connectors, or any other configuration of electrical contacts which engage when the cartridges 10, 20 are connected to the control unit 2.

A user may adjust the relative amounts of vapor generated by each of the cartridges 10, 20, and hence the relative visibility of the vapor generated by the e-cigarette as a whole, during use. For example, if the user wants to generate a vapor with maximum visibility, the user may configure the electronic cigarette to generate vapor from the first source liquid in the first cartridge. Conversely, if the user wants to generate a vapor with minimum visibility, the user may configure the electronic cigarette to generate vapor from the second source liquid in the second cartridge. Furthermore, in some implementations the user may be able to generate a vapor with a visibility between the visibility associated with the first cartridge and the visibility associated with the second cartridge by controlling the electronic cigarette to generate vapor from both cartridges in a selected ratio/proportion. It will be appreciated that the higher the proportion of the overall vapor generated by the first cartridge to the vapor generated, the higher visibility/opacity of the resultant vapor (i.e. supplying relatively more power to the first cartridge will result in a relatively more visible vapor while supplying relatively more power to the second cartridge will result in a relatively less visible vapor).

Thus, the user may configure the electronic cigarette to generate vapor having a desired/selected degree of relative visibility by configuring the electronic cigarette to generate vapor from the respective cartridges in proportions corresponding to the desired/selected degree of relative visibility. In this sense, it will be appreciated that the user will typically not control the device with an expectation of providing a vapor with a specific degree of opacity, but rather will simply control the device to provide what the user subjectively considers to be more visible vapor or less visible vapor according to their wishes at a particular time.

Thus, a greater degree of vapor visibility may be obtained by configuring the electronic cigarette to generate a higher proportion, or all, vapor from the first cartridge, whereas a lower degree of vapor visibility may be obtained by configuring the electronic cigarette to generate a higher proportion, or all, vapor from the second cartridge.

The electronic cigarette may, for example, be configured to provide a desired degree of visibility through a configuration menu for the device, which may be accessed through the user input button 34. For example, a user may press the button 34 in a predefined sequence to enter a programming mode, and then press the button in a further predefined sequence to set desired vapor generation levels for respective ones of the cartridges (and hence the overall visibility of vapor generated by the device). The user may be able to freely set the relative amounts of vapor (i.e. level of power supplied) for each cartridge and/or may select from a number of predefined settings depending on the implementation at hand. In other examples there may be other means for setting the relative levels of vaporization from the two cartridges, for example the device may comprise additional user inputs, for example one or more additional buttons/dials/sliders/touch screen for this purpose, or the device may support remote programming using an ancillary device arranged to exchange data with the electronic cigarette 1, for example a computing device, such as a smartphone, running an appropriate application. More generally an algorithm may be used to supply relative amounts of power to balance the amount of vapor from each cartridge based on a user's preference(s), which could be stored in a memory.

In some cases the relative levels of vaporization may not be defined in advance of a given puff, but may be selected in real-time during use, for example by having a separate activation button associated with each of the cartridges which may be independently activated by a user, for example on a puff-by-puff basis. Thus, a user may press one button to select vapor generation from one cartridge, or press the other button to select vapor generation form the other cartridge, or press both buttons simultaneously to select vapor generation from both cartridges. The device may be configured so that when both buttons are activated, the vaporizers associated with the respective cartridges are supplied with around half the power than would be the case when only one button is activated so the overall amount of vapor generated (i.e. the overall amount of precursor material that is vaporized in a given time) remains broadly the same regardless of how many cartridges are simultaneously activated. It will be appreciated in other example implementations the relative proportions associated with each cartridge may be different when a user presses both buttons, for example the power to each cartridge may be split on a 60-40 basis, or in accordance with any other ratio, when both buttons are pressed. Furthermore, this ratio may be predefined or configurable by a user. Furthermore still, the total power supplied to the cartridges (i.e. the sum of the powers supplied to both cartridges) may be varied to allow for an overall variable power setting for the device. Power may be supplies to both cartridges at the same time, or overlapping times, or in a time-multiplex manner.

Thus, in accordance with the principles described herein, a user may configure the electronic cigarette 1 to generate a vapor using predominantly (or exclusively) the first vapor precursor material to generate a vapor with relatively high visibility, or may configure the electronic cigarette to generate a vapor using predominantly (or exclusively) the second vapor precursor material to generate a vapor with relatively low visibility, or may configure the electronic cigarette 1 to generate a vapor using an intermediate mixture of the two vapor precursor materials to generate a vapor pour having intermediate visibility. In some implementations the user may be provided with the opportunity to selectively power the heater in the first cartridge to provide relatively high visibility vapor or the heater in the second cartridge to provide less-visible vapor, but the electronic cigarette 1 might be configured to not allow power to be supplied to both heaters simultaneously. That is to say, the ratios for vapor generation that may be selected by a user in some cases might only be 100:0 or 0:100 with nothing in between. In some implementations in addition to the control circuitry being configured to allow different amounts of relative vapor generation from the different vapor precursor materials to provide vapors with correspondingly different visibilities, the control circuitry may also be configured to allow for different overall amounts of vapor generation for a given ratio. That is to say, a user may be able to adjust the overall total power supplied to the cartridges, as well as the ratio in which the power is supplied to the respective cartridges.

As noted above, there are various ways in which the relative amounts (and in some cases the total amount) of vapor to be delivered by the cartridges may be pre-configured by a user to provide a vapor having a desired/selected degree of visibility. When the electronic cigarette has been configured in this way and is in its normal operating mode a user may then simply press the button 34 to activate the respective heaters 15, 25 in the respective cartridges 10, 20 in accordance with the configured power settings.

Accordingly, when a user presses the user input button 34 the vapor generation function of the electronic cigarette 1 is activated—i.e. electrical power is supplied to one or other or both of the heaters 15, 25 in accordance with a selected configuration. Although in this example implementation a user input button 34 is used to trigger vapor generation, it will be appreciated that the activation of vapor generation may be based on other techniques. For example, instead of using a button to activate the supply of power to the heaters, an inhalation sensor, for example based around a pressure sensor/microphone arranged to detect a drop in pressure when a user inhales on the device, may be used.

When the vapor generation function of the electronic cigarette 1 is activated, a user sucks/inhales on the mouthpiece outlet 8 of the mouthpiece cover 6 to draw air through the electronic cigarette. The flow of air through the electronic cigarette is schematically indicated in FIG. 1 by a series of arrows. Thus, air is drawn from the environment into the electronic cigarette 1 through one or more air inlets 7, which in this case are provided in the control unit 2. A portion of the air drawn into the electronic cigarette 1 is drawn along an inlet air path to enter the vaporization chamber 16 in the first cartridge 10 and a portion of the air drawn into the electronic cigarette 1 is drawn along an inlet path to enter the vaporization chamber 26 of the second cartridge 20. Accordingly, the incoming air flows past the respective heaters 15, 25 in the respective vaporization chambers 16, 26 while one or both of the heaters is receiving electrical power from the battery in the control unit 2 so as to generate a vapor from the relevant source liquid(s)/vapor precursor material in the corresponding vaporization chamber(s). The vaporized precursor material is then incorporate/entrained into the airflow and drawn through the relevant cartridge (along the air path defined by the gap between the flat of the reservoir and the outer housing discussed above) to exit the relevant cartridge through its opening 19, 29 and into the mixing chamber 5, from where it is drawn out of the mouthpiece opening 8 for inhalation by a user.

Thus, in accordance with certain embodiments, an electronic cigarette may be configured to allow a user to selectively generate vapors with different degrees of visibility according to the user's wishes, for example to allow the user to generate less visible vapor in public than when at home. It will of course be appreciated that the specific reasons why and circumstances under which a user might prefer to generate a visible vapor or less-visible vapor are not significant to the principles described herein. In some implementations a switch between different degrees of vapor generation may be made with a degree of automation. For example the electronic cigarette may include a timer and be configured so as to generate less-visible vapor when used during certain times (e.g. user-configured working hours) and to generate more-visible vapors at other times. In another example, the electronic cigarette may include a GPS receiver and be configured so as to generate more-visible vapor when used in certain locations (e.g. a user-configured home location) and to generate less-visible vapors in other locations.

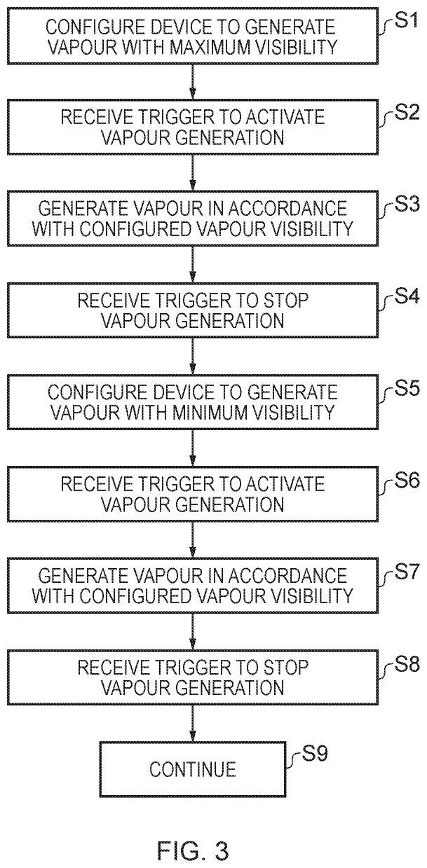

FIG. 3 is a flow chart schematically representing some operational steps for the vapor provision system 1 represented in FIGS. 1 and 2 in accordance with an embodiment of the present disclosure. The control circuitry 36 is configured to implement this processing in accordance with conventional programming/processing techniques. For the sake of this example it is assumed a user initially wishes to generate vapor with maximum visibility, but after using the device to generate vapor with maximum visibility wishes to switch to generating vapor with minimum visibility. In this example it is assumed that the device is configured to generate vapor by supplying a fixed amount of power P to the heaters when generating vapor during the session of use represented in FIG. 3, wherein the fraction of the total power P supplied to each heater is dependent on the desired visibility configuration. The total power P for vapor generation may be a fixed characteristic of the electronic cigarette, or a user configurable setting. If the total power P is user-configurable, it may, for example be changed on a puff-by-puff basis.

Thus, in S1 the user configures the electronic cigarette 1 for generating vapor with the highest possible visibility, i.e. by supplying the total power P only to the first cartridge when vapor is to be generated. As discussed above, this configuration step may be achieved by programming the device using the input button 34 or other means.

In S2 the control circuitry 36 receives an indication (trigger) that the vapor generation function of the electronic cigarette should be activated. This trigger indication may, for example, correspond with a detection that the user has pressed the user input button 34 while the device is in a normal operating mode. Alternatively, and depending on implementation, S2 may be based on the detection of a different type of trigger to activate vapor generation, for example a pressure sensor-based detection indicating that a user has started to inhale on the device may be used to provide what is in effect an automatic trigger to start vapor generation (i.e. supply power to the heater(s)). This step may be performed in accordance with conventional techniques for detecting when to activate vapor generation in a vapor delivery system such as an electronic cigarette.

In S3, the control circuitry drives the heater(s) in the respective cartridges in accordance with the desired vapor visibility setting, which in this example corresponds with supplying the total power P to the first vaporizer in the first cartridge (i.e. the cartridge containing the vapor precursor material associated with the higher visibility vapor). It will be appreciated that the ratio of power provided to the respective cartridges in S3 will depend on the desired visibility configuration established in S1. Thus, in S3 the user is provided with vapor in accordance with the selected visibility configuration established in S1 so that when the user exhales the vapor generated in S3, the resulting cloud/plume has a relatively high visibility/opacity (low transparency).

In S4, while generating vapor based on the desired visibility discussed above in relation to S3, the control circuitry 36 receives an indication (trigger) that the vapor generation function of the electronic cigarette should be stopped. This trigger indication may, for example, correspond with a detection that the user has released the user input button 34. Alternatively, and depending on implementation, S4 may be based on the detection of a different type of trigger to stop vapor generation, for example a pressure sensor-based detection that a user has stopped inhaling on the device may be used to provide what is in effect an automatic trigger to stop vapor generation. This may be performed in accordance with conventional techniques for detecting when to stop vapor generation in a vapor delivery system such as an electronic cigarette. In response to the trigger to stop vapor generation being received in S4, the control circuitry cuts the supply of electrical power to the relevant heater(s). This represents the end of a puff on the device.

In S5 it is assumed that the user has changed their mind in respect of the degree of visibility for the vapor they would like the electronic cigarette to generate, and in particular, in this example, it is assumed that the user wishes to switch to generating vapor having minimum visibility. This may be, for example, because the user has entered a public place and would prefer to vape more discreetly, but it will be appreciated that the particular reason why the user wishes to change vapor visibility is not significant to the principles described herein. Thus, in S5, the user configures the electronic cigarette for generating vapor with the lowest possible visibility, i.e. by supplying the total power for vapor generation P only to the second cartridge when vapor is to be generated. As discussed above, this configuration step may be achieved by programming the device using the input button 34 or other means. As already noted, it is assumed that the total power for vapor generation P remains constant throughout the session of use represented in FIG. 3, but in other cases the user may also choose to adjust the total power P for the electronic cigarette to use for vapor generation at this, or indeed any other, stage.

In S6 the control circuitry 36 receives an indication (trigger) that the vapor generation function of the electronic cigarette should be activated, for example because the user has pressed the user input button 34. This step corresponds with, and will be understood from, S2 discussed above.

In S7, the control circuitry drives the heater(s) in the respective cartridges in accordance with the current desired vapor visibility setting, which in this example corresponds with supplying the total power P to the second vaporizer in the second cartridge (i.e. the cartridge containing the vapor precursor material associated with the lower visibility vapor). Thus, in S7 the user is provided with vapor in accordance with the selected visibility configuration established in S6 so that when the user exhales the vapor generated in S7, the resulting cloud/plume has a relatively low visibility/opacity (i.e. high transparency).

In S8, while generating vapor based on the desired visibility discussed above in relation to S7, the control circuitry 36 receives an indication (trigger) that the vapor generation function of the electronic cigarette should be stopped. This corresponds with, and will be understood from, S4 discussed above.

Thus, in accordance with the instance described herein, the user is able to readily and easily generate vapor with a selectable degree of visibility. As already mentioned, in some examples the vapor generation may not come exclusively from one or other cartridge, but may comprise a mixture of vapor generated from both cartridges at the same time.

As schematically indicated in S9, the electronic cigarette may continue in a standby mode awaiting the user's next puff or change of configuration setting so that is can respond accordingly in line with the principles described herein. When a user has completed a session of using the electronic cigarette, it may be switched off until the next time a user wishes to use the device. When the electronic cigarette is switched on again it may retain the configuration setting it had before being switched off, adopt a default configuration setting (e.g. always start in minimum vapor visibility mode), or require the user to configure a desired visibility setting before use.

While some particular examples have been described above, it will be appreciated that there are many modifications that could be made in accordance with other implementations.

For example, in the implementation represented in FIGS. 1 and 2, each of the cartridges comprises its own vaporizer. That is to say, there is a separate vaporizer associated with each of the vapor precursor materials. In this case the control circuitry 36 may generate a vapor from a selected ratio of the first source liquid 12 and the second source liquid 22 by applying an appropriate amount of power to the heaters of the respective vaporizers. This results in the generation of initially separate vapors from each of the different source liquids, with these vapors then being combined/mixed in the mixing chamber 5 before inhalation by a user. That is to say, in this example implementation, the vapors are mixed in the desired ratio after generation. However, in other examples a first source liquid and a second source liquid may be mixed in a desired ratio prior to vaporization, for example by delivering the first source liquid and the second source liquid to a single vaporizer at relative rates corresponding to a desired ratio. In such a case, the single vaporizer may, for example, be provided within a control unit part of a vapor provision system. The different source liquids may be delivered to the vaporizer at the desired rates/in the desired ratio using appropriately controlled pumps or valves. For example, if a user indicates a desire to use twice as much of a first source liquid than a second source liquid to generate a vapor with a desired degree of visibility, the control circuitry may be configured to pump liquid from a reservoir for the first source liquid at a rate which is twice the rate at which liquid is pumped from a reservoir for the second liquid. It will, of course, be appreciated that the control circuitry may also be configured to pump liquid exclusively from one or other reservoir to generate vapor using only one of the first or second paper precursor material according to a configuration setting for the device.

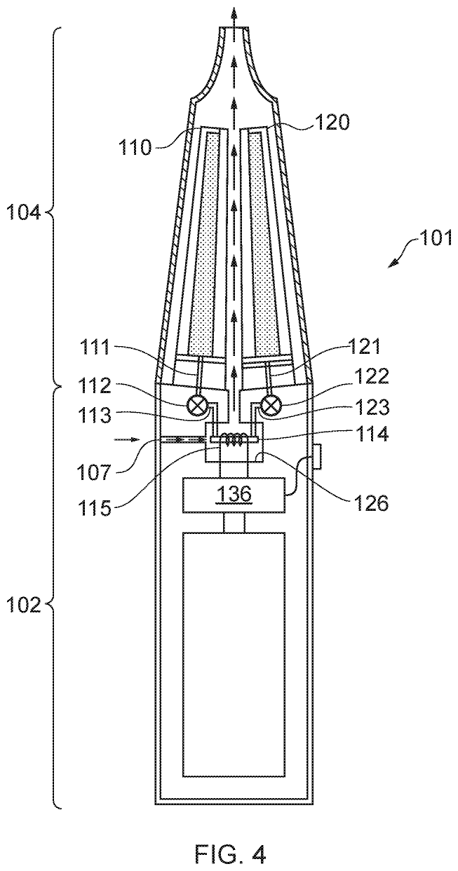

FIG. 4 is a schematic cross-section of a vapor provision system 101 according to certain embodiments of the disclosure that uses a single vaporizer. Many aspects of the system 101 represented in FIG. 4 are similar to, and will be understood from, corresponding aspects of the system 1 represented in FIG. 1, and these are not described again in the interest of brevity. The system of FIG. 4 again comprises a vapor generation assembly 104 including a first source liquid cartridge 110 and a second source liquid cartridge 120 and a control unit part 102 comprising control circuitry 136 (and other elements such as discussed above). However, the system 101 of FIG. 4 differs from the system 1 of FIG. 1 in having a single vaporizer comprising a wick 114 and a heater 115, rather than separate vaporizers in each cartridge. This single vaporizer may be based on the same principles as described above for the separate vaporizers in each of the cartridges, or indeed may be based on any vaporization technology. In the example of FIG. 4, the single vaporizer is mounted in the control unit 102 part of the system 101. The first cartridge 110 and second cartridge 120 contain respective source liquids, which may be the same as discussed above for the system 1 represented in FIG. 1. However, the respective cartridges do not comprise a vaporizer and ceramic disc, but instead each comprise a fluid path 111, 121 providing fluid communication between their respective reservoirs of source liquid and the controller unit 102.

When the respective cartridges 110, 120 are coupled to the control unit 102, their respective fluid paths 111, 121 align with corresponding fluid paths 113, 123 in the control unit 102. The fluid paths 113, 123 in the control unit each comprise a micro fluid pump 112, 122, which may be based on any known technology, and provide for pumped fluid communication between the fluid paths 111, 121 in the cartridges and the wick element 114 of the vaporizer in the control unit 102.

The control circuitry 136 is configured to control the respective fluid pumps 112, 122 to deliver liquid from the respective cartridges to the wick 114 of the vaporizer via the respective fluid paths 111, 113, 121, 123. The control circuitry 136 is also configured to drive the heater 115 of the vaporizer in the control unit 102 to generate vapor from the combination of liquid delivered to the wick 114 by the respective pumps 112, 122. The vapor is generated in a vapor generation chamber 126 so that when a user draws on the mouthpiece end of the system 101, air is drawn in through an inlet 107 in the control unit 102, into the vapor generation chamber 126 where it mixes with the vaporized mixture/combination of source liquids and is drawn out through the mouthpiece of the system as schematically indicated by the arrows in FIG. 4.

The control circuitry 136 can configure the pumping rates of the respective pumps 112, 122 so as to deliver source liquid for vaporization from the respective cartridges in a desired/selected combination in much same way as the control circuitry 36 of the example implementations discussed above can control the relative amounts of power delivered to the respective vaporizers in the respective cartridges in that example. Thus, the relative rates of fluid delivery to the vaporizer may be modified in response to user input to modify a visibility characteristic associated with the resulting vapor.

More generally, it will be appreciated that the specific manner in which the vapor is generated, both in terms of the underlying vapor generation technology, and whether the source liquid is in effect mixed/combined before or after vaporization is not significant to the underlying principle of providing vapors with a user-selectable degree of visibility.

Thus it will be appreciated that whereas the above-described embodiments have primarily focused on an electrical heater based vaporizer for heating a source liquid, the same flavor modification principles may be adopted in accordance with vaporizers based on other technologies, for example piezoelectric vibrator based vaporizers, and devices based on other vapor precursor materials, for example gels or solid materials, such as plant derived materials, such as tobacco derivative materials.

It will also be appreciated that while the above-described examples have focused on implementations comprising two vapor precursor materials, in other implementations, the same principles may be applied in respect of vapor provision systems comprising more than two separate vapor precursor materials. For example, a vapor provision system in accordance with some implementations may comprise three, four or more different vapor precursor materials to provide for a wider range of vapor characteristics, for example in terms of visibility.

The above-examples have focused on implementations comprising multiple vapor precursor materials that may be independently vaporized for inhalation. That is to say, vapor for inhalation can be generated from one or other or both vapor precursor materials based on a desire to degree of vapor visibility. However, in some other implementations there may be a primary vapor precursor material which is always used and which generates a relatively low-visibility vapor and which includes any desired additives, such as flavoring and/or nicotine, and a secondary vapor precursor material which is provided purely to boost vapor visibility by mixing vapor from the secondary vapor precursor material with vapor from the primary vapor precursor material, but which is not intended to be used independently. For example, the primary vapor precursor material may comprise a broadly conventional electric cigarette formulation, whereas the secondary vapor precursor material may comprise pure vegetable glycerol or another material associated with the generation of vapors with relatively high optical density.

Thus, there has been described a vapor provision system configured to generate vapor with a selectable degree of visibility for inhalation and subsequent exhalation by a user; wherein the system comprises: a first vapor precursor material for generating a vapor having a first degree of visibility; a second vapor precursor material for generating a vapor having a second degree of visibility, wherein the first degree of visibility is greater than the second degree of visibility such that vapor generated from the second vapor precursor material is less visible than vapor generated from the first vapor precursor material; at least one vaporizer operable to generate vapor from the first vapor precursor material and the second vapor precursor material; and control circuitry configured to control the at least one vaporizer to generate vapor from the first vapor precursor material and the second vapor precursor material in a selectable ratio to provide a vapor having a correspondingly selectable degree of visibility.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future.

1. A vapor provision system configured to generate vapor with a selectable degree of visibility for inhalation and subsequent exhalation by a user, the system comprising:
a first vapor precursor material for generating a vapor having a first degree of visibility;
a second vapor precursor material for generating a vapor having a second degree of visibility, wherein the first degree of visibility is greater than the second degree of visibility such that vapor generated from the second vapor precursor material is less visible than vapor generated from the first vapor precursor material;
at least one vaporizer operable to generate vapor from the first vapor precursor material and the second vapor precursor material by heating the first vapor precursor material and the second vapor precursor material; and
control circuitry configured to control the at least one vaporizer to generate vapor from the first vapor precursor material and the second vapor precursor material in a selected ratio to provide a vapor having a correspondingly selected degree of visibility.

2. The vapor provision system of claim 1, wherein the control circuitry is configured to control the at least one vaporizer to generate vapor from the first vapor precursor material and the second vapor precursor material in a selected ratio by selectively generating vapor from only one or the other of the first vapor precursor material or the second vapor precursor material.

3. The vapor provision system of claim 1, wherein the control circuitry is configured to control the at least one vaporizer to generate vapor from the first vapor precursor material and the second vapor precursor material in a selected ratio by generating vapor from a selected mixture of the first vapor precursor material and the second vapor precursor material.

4. The vapor provision system of claim 1, wherein the selected ratio is based on input received from a user.

5. The vapor provision system of claim 1, wherein the at least one vaporizer comprises a first vaporizer operable to generate vapor from the first vapor precursor material and a second vaporizer operable to generate vapor from the second vapor precursor material.

6. The vapor provision system of claim 1, wherein the at least one vaporizer comprises a single vaporizer operable to generate vapor from both the first vapor precursor material and the second vapor precursor material.

7. The vapor provision system of claim 1, wherein the first vapor precursor material comprises a first base liquid formulation and the second vapor precursor material comprises a second base liquid formulation, wherein the first base liquid formulation and the second base liquid formulation are different.

8. The vapor provision system of claim 7, wherein the first base liquid formulation and the second base liquid formulation comprise one of: different components, or the same components in different concentrations.

9. The vapor provision system of claim 1, wherein the first base liquid formulation contains a greater concentration of glycerol than the second base liquid formulation.

10. The vapor provision system of claim 9, wherein the concentration of glycerol in the first base liquid formulation is greater than the concentration of glycerol in the second base liquid formulation by at least a factor selected from the group consisting of: 1.2, 1.3, 1.4, 1.5, 2, 2.5, 3, 3.5, 4, 4.5 and 5.

11. The vapor provision system of claim 1, wherein the second base liquid formulation contains a greater concentration of propylene glycol than the first base liquid formulation.

12. The vapor provision system of claim 11, wherein the concentration of propylene glycol in the second base liquid formulation is greater than the concentration of propylene glycol in the first base liquid formulation by at least a factor selected from the group consisting of: 1.2, 1.3, 1.4, 1.5, 2, 2.5, 3, 3.5, 4, 4.5 and 5.

13. The vapor provision system of claim 1, wherein the first degree of visibility is greater than the second degree of visibility by at least a factor selected from the group consisting of: 1.5, 2, 3, 4, 5, 6, 7, 8, 9, and 10.

14. The vapor provision system of claim 1, wherein the system is a modular system comprising a control unit, a first replaceable cartridge and a second replaceable cartridge, wherein the control unit comprises the control circuitry, the first cartridge comprises the first vapor precursor material, and the second cartridge comprises the second vapor precursor material.

15. Vapor Vapour provision means for generating a vapor with a selectable degree of visibility for inhalation and subsequent exhalation by a user comprising:
a first vapor precursor material adapted for generating a vapor having a first degree of visibility;
a second vapor precursor material for generating a vapor having a second degree of visibility, wherein the first degree of visibility is greater than the second degree of visibility such that vapor generated from the second vapor precursor material is less visible than vapor generated from the first vapor precursor material;
vaporizing means for generating vapor from the first vapor precursor material and the second vapor precursor material by heating the first vapor precursor material and the second vapor precursor material; and
control means for controlling the vaporizing means to generate vapor from the first vapor precursor material and the second vapor precursor in a selected ratio to provide a vapor having a correspondingly selected degree of visibility.

16. A method of generating a vapor with a selectable degree of visibility for inhalation and subsequent exhalation by a user the method comprising:
providing a vapor provision system comprising:
a first vapor precursor material for generating a vapor having a first degree of visibility,
a second vapor precursor material for generating a vapor having a second degree of visibility, wherein the first degree of visibility is greater than the second degree of visibility such that vapor generated from the second vapor precursor material is less visible than vapor generated from the first vapor precursor material, and
at least one vaporizer operable to generate vapor from the first vapor precursor material and the second vapor precursor material; and
controlling the at least one vaporizer to generate vapor from the first vapor precursor material and the second vapor precursor in a selected ratio to provide a vapor having a correspondingly selected degree of visibility by heating the first vapor precursor material and the second vapor precursor material.

17. (canceled)

18. (canceled)
